(19) United States
(12) Patent Application Publication
(10) Pub. No.: US 2013/0231639 A1
(43) Pub. Date: Sep. 5, 2013

(54) Drainage Catheter

(71) Applicant: Dean Tatlow, Calgary (CA)

(72) Inventor: Dean Tatlow, Calgary (CA)

(21) Appl. No.: 13/561,018

(22) Filed: Jul. 28, 2012

(30) Foreign Application Priority Data
Mar. 3, 2012 (CA) 2770277

(51) Int. Cl. A61M 27/00

(52) U.S. Cl. 604/543; 604/544

(57) ABSTRACT

A medical catheter inserted into a body cavity for drainage of fluid. The catheter comprises flexible fibers/filaments that form a plurality of channels for movement of fluid. These fibers/filaments are located around a central inflation device contained within a flexible tubular catheter, composed of two parts. The outer sleeve and inner sleeve form a tubular catheter joined by a flexible connector that interlocks the sleeves during insertion. Once inserted the connection between the two sleeves is released, enabling the outer sleeve to move over top of the inner sleeve revealing fibers/filaments for fluid drainage. An inflatable retention balloon contained within the fibers/filaments secures the catheter in the bladder once inflated. This arrangement allows insertion of non-contaminated fibers/filaments into the body cavity. Insertion of these fibers/filaments creates a more complete drainage of fluid, decreased risk of bacterial infection, reduction of blockage, and improved patient comfort.

### BACKGROUND OF THE INVENTION

1. Field of the Invention

This invention provides an improved catheter, more particularly of the type used in urinary, chest tube, and wound catheters.

2. Description of the Prior Art

Catheters are commonly used in the medical system by a patient who is unable to urinate. There are many reason why a patient may not be able to urinate; from being bedridden, to being in a diseased state, to having structural abnormalities. A urinary catheter allows a patient to successfully empty the bladder though urinary tract infections are a common result. When the catheter is inserted into the urethra and bladder, bacteria may be passed into the patient. Microbes may develop on the external surface of the catheter if it is not properly sterilized or it is mishandled by the patient or patient's care giver.

Catheters are usually designed with a flexible hollow tube that is inserted into the body to provide a channel for fluid to pass. The tube typically contains one or more lumens within which the fluid may flow.

There are two types of catheters: intermittent and indwelling. An intermittent catheter is designed for short term use as it is inserted before each urination and then removed; an indwelling catheter is designed to remain within the patient for a long period. Frequent insertion of the intermittent catheter is uncomfortable to the patient hence, such a catheter is not an effective solution for a patient who frequently suffers from a bladder deficiency. An indwelling catheter requires less replacement and can be used over a longer period of time. A very commonly used indwelling catheter in the medical system is the Foley catheter. The Foley catheter, designed in 1930s, is a rubber tube with a balloon tip. This indwelling catheter comprises two separate tubes or lumens running down a long tubular shaft. One lumen is open at both ends and allows urine to be drawn, the other end is connect to a balloon at the tip. After insertion into the bladder via the urethra, a syringe fills the balloon tip with sterilized liquid or air, in order to prevents the catheter from passing back through the bladder neck. This type of catheter allows for drainage of urine from the bladder. An example of this catheter is described in international patent application PCT/SE82/00124. All catheters discussed going forward are indwelling.

There are many limitations of prior art catheters such as the Foley. A residual volume of urine is left in the bladder since the drainage aperture is located above the retention balloon. This residual volume of urine is thought to contribute to bladder infections. Furthermore, the size of a Foley type catheter is generally large to best accommodate the inflation and draining lumen, and thus are difficult to insert and uncomfortable to the patient. Finally, when positioned inside the bladder the retention balloon rests on the most sensitive area of the bladder, the trigone region and bladder neck. This can cause increased discomfort to the patient.

U.S. Pat. No. 6,558,350 B1 uses a woven mesh to form at least a portion of the elongated tube and retention member. The woven mesh is permeated by fluid and therefore facilitates drainage of fluid into the elongated tube of the catheter. One disadvantage of this design is that the mesh may become accumulated with cellular debris and therefore easily blocked. The urethra is lined with an urothelial type epithelium. Mucous secreting cells frequently occur in the stratified epithelium and there are numerous recesses connected to mucous glands. Secretion of mucus may cover the mesh layer decreasing the permeability of the mesh for fluid and fluid may be unable to drain. A further concern with this device is the slow drainage of fluid, causing more fluid to enter the bladder then leave. Finally, with this device the mesh material may provide a scaffolding for new growth of tissue, mainly by epithelium cells, which eventually may incorporate the mesh into the surrounding area resulting in difficult removal.

U.S. Pat. No. 7,264,609 describes a catheter that eliminates the problem of residual urine and of bladder wall irritation caused by continuous contact with a spherical retention balloon. The catheter contains longitudinal strips in the proximal end forming retention wings and has a widen space for a drainage aperture. The retention wings reduce the area of bladder-catheter contact within the trigone and bladder neck region, decreasing bladder wall irritation. U.S. Pat. No. 7,264,609 has a plurality of drainage apertures extending distally to the cysto-urethral junction entering a common collection tube, and it is this tube that may become blocked. In addition, the common collecting duct does not allow for the normal passage of urine to flush debris through the urethra. The catheter can leave spaces between the urethra and catheter where urine can collect and pose risk for infection to the patient.

U.S. Pat. No. 7,662,146 details a catheter surrounded by a protective sheath. The protective sheath serves two purposes; to protect the catheter from contamination; and to contain wetting agents used to lubricate the catheter. The invention eliminates the need for multiple steps required in the sanitation and lubrication of catheters prior to insertion. Insertion of a sterilized tubular catheter, however, does not eliminate infection from other sources. Cellular debris still commonly blocks the catheter resulting in an stagnant environment precluding infection.

Methods to prevent blockage are shown in U.S. Pat. No. 4,755,175 which describes a catheter where the interior of the tubular insertion section is arranged to support a sieve or filter element. This creates turbulence in fluid flow so as to prevent blockage. Additionally, U.S. Pat. No. 5,201,724 describes a catheter with a biodegradable portion which resists infection or the formation of blockages. Solid biodegradable material is released as a result of bio degradation such that a controlled and continuous release of bio degradation products takes place through-out the said period of insertion preventing blockage. The disadvantage of both these patents is the potential for mucous secretions to encapsulate the devices and hence limit their function.

The current invention has numerous benefits over existing catheters. With many existing catheters, patients must be constantly monitored for blockage caused by urinary secretions. When a catheter becomes blocked, urine is unable to escape the bladder which may lead to bacterial infection. Blockage is less likely with the current invention because of the plurality of channels and the ability for fluid to transfer amongst the bundles of fibers. Even in catheter's that use a sieve element or biodegradable end, blockage may still occur because of mucus secretions. In other existing catheters, problems arise because bacteria may develop between the catheter wall and urethra, causing infection. In the current invention the arrangement of fiber bundles allows fluid to flow through the urethra and permits flushing of the urethral wall. The current invention also allows for the insertion of non-contaminated fibers into the urethra and bladder. The current invention uses flexible fibers/filaments to create a fluid drainage catheter to minimize the chance of obstruction and decrease the risk of infection in a patient as a result of incomplete drainage and irritation. The invention is an indwelling urinary tract catheter that increases patient comfort once inserted into the urinary tract. There is a need for a leaner, more flexible, convenient and easier to handle catheter; one which reduces infection risk and decreases blockage. The proposed invention addresses these needs.

### SUMMARY OF THE INVENTION

The current invention is unique in its assembly. Fibers are arranged to create a plurality of channels which drain fluid from the body cavity. The device includes an outer and inner sleeve forming an elongated tube, enclosing fibers which surround an inflation device. Fibers run all or part, in a longitudinal direction, and are braided, weaved or wrapped around the inflated device such that expansion of the retention balloon cause fibers to adapt to configuration of the inflated retention balloon. The inner sleeve contains two projections which help fix or anchor the braided fibers and inflation device by constricting movement. The outer sleeve is designed to slide over the inner sleeve and prevent contamination of braided fibers and the inflation device by handling of the catheter during insertion. At the distal end of the inner sleeve the catheter bifurcates creating an area where syringe and collection vessels/irrigation connectors are located.

An objective of the current invention is to facilitate the insertion of sterilized braided fibers that contain a retention device, into a canal or canal-like organ. This minimizes the likelihood that an infection will occur in the patient as the result of insertion and subsequent operation of the catheter. The current invention is a drainage catheter composed of non-contaminated flexible fibers surrounding an inflation device enclosed within an elongated tube with two parts, the outer and inner sleeves. After insertion the outer sleeve may be removed to reveal the non-contaminated fibers and inflation device.

Various literary articles and patent applications have disclosed catheters for the elimination of fluid. An example of a very common catheter is the Foley catheter. The main disadvantage of the Foley catheter is that it is not capable of fully eliminating fluid from the bladder. Instead, a residual volume of urine collects between the bladder neck and the opening of the catheter which resides above the retention balloon. This residual volume of urine has been linked to urinary tract infections in catheterized patients. As the elimination of urine from the bladder is imperative, there is a great need for a simple, convenient, inexpensive, and commercially viable catheter. A principle objective of the current invention is a more complete elimination of urine from the bladder. The present invention, with a length of braided fibers extending past the retention balloon, can drain urine from the base of the bladder reducing or eliminating residual urine. Another advantage of the current invention is its ability to effectively eliminate urine from a patient while in various positions unlike existing catheters where the patient needs to be in an upright position for optimal drainage. The length of braided fibers extending past the retention balloon may move with the patient such that it is always in a preferable position for urine drainage.

As previously noted, the Foley catheter is particularly uncomfortable to the patient due to its size and inflexibility. The present invention is composed of flexible fibers braided around a very fine inflation tube, decreasing the need for a large drainage lumen, as in the Foley catheter. Furthermore, the flexible fibers can adapt to the curvature of the urethra and provide increased comfort for the patient.

The current invention possesses similar properties to inventions which decrease contact with a spherical retention balloon in the trigone region of the bladder. Previous inventions use a wing designed catheter to decrease pressure on the trigone region. The present invention has braided fibers which mimic the winged catheter by having strips of fibers wrapped around the retention balloon. This invention prevents pressure on the trigone region of the bladder by distributing it to the fibers, helping to prevent discomfort caused by the retention balloon. An added feature of the current invention is the ability to release tension on the fibers when a patient is lying down allowing the retention balloon to fall off the trigone region. This can be accomplished by pressing on the inner sleeve anchor area which causes the two projections to release, allowing for movement of the braided fibers and inflation device.

Another advantage of the current invention is the absence of a common collection tube which may become blocked with cellular debris. The current invention is composed of an arrangement of fibers which create a plurality of channels and allows fluid to flow between and amongst the fibers. The length of the fibers within the bladder and the arrangement of these fibers substantially decrease the chance of catheter obstruction, by providing alternate routes of fluid flow if a channel or channels are blocked. The current inventions braided fibers, decreasing the likelihood of mucus clogging the channels and increasing the area for absorption of fluid.

A potential problem with the mesh layer catheter is the transfer of fluid by direct contact with clothing or other apparel outside the body. The shield in the current invention prevents direct contact with fibers outside the body.

Another difference between the two inventions is the mesh material is fully collapsible unlike the present invention where multiple fibers are wrapped around the inflation device. The current invention provides longitudinal filament that are wrapped around a central inflation device. The longitudinal fibers create fine tubes for transportation of fluid, whereas the previous invention uses a mesh material with no directionality or substantial depth. The current invention not only prevents clogging but increases flexibility, decreases size, and has a more simple design.

A problem with Foley type catheters is bacteria may occur in spaces between the catheter tube and urethra wall resulting in infection. Although, catheter associated bacterial infection is not fully understood, it is generally believed that migration of bacteria extraluminally in the periurethral space may provide a pathway of infection. A benefit of the current invention is the arrangement of fibers permits movement of body fluid in and around fibers cleansing the urethra wall by flushing bacteria and debris out of the body. Additionally, fibers/filaments may be coated to prevent formation of biofilms which may also lead to infection. Furthermore, the current invention can be irrigated through the flow of disinfecting or sterilized solutions along the outside surface of the fibers supplied by the irrigation port. This prevents stagnation and obstruction of the cellular debris. The internal shield of the current invention is designed to eliminate contact with the fibers outside the catheter and prevent possible contamination.

A particular problem that affects balloon catheters when deflated, is cuffing, where the balloon may incur crease or ridge formations. These areas of the balloon may also become encrusted with a build-up of mineral deposits making removal of the catheter more difficult. The ridges and encrusted material can cause scratching of the urethral walls or bleeding. It becomes apparent when a care giver tries to removes a catheter that a cuff has formed with balloon deflation, since it appears to become ‘stuck’. At this point a patient may be referred to the urology unit for catheter removal. The current invention decreases the impact of cuffing caused by the collapsed balloon and therefore reduces the force required to remove the catheter. The retention balloon may be further deflated, and the impact of cuffing reduced by pulling the inflation device into the weave of the fibers, protecting the walls of the urethra. Fibers would further encapsulate over the balloon creating a smooth area for catheter removal, reducing the need for a urology referral.

To prevent the catheter from being unintentionally inflated within the urethra the outer sleeve is not withdrawn until urine is seen exiting the catheter indicating the proper position for the catheter has been achieved. Once this position has been achieved, the catheter will be further inserted into the bladder for an appropriate distance. The outer sleeve is then removed and the retention balloon remains located inside the bladder.

The current invention can be lubricated to improve patient comfort. At least a portion of the fiber material, preferably the area in between the proximal and distal end, engages the internal mucous membrane of the urethra when provided with a soft foam coating. Fiber material could also be covered with an antimicrobial gel.

The invention provides a completely new approach to draining fluid from body compartments, wherein fibers/filaments material lies within the body allowing movement of fluid outside the patient.

It is the objective of the current invention to improve catheter performance by decreasing infection risk, improving patient comfort, and reducing catheter blockage. Infection risk is decreased by inserting sterilized fibers, eliminating residual urine, reducing irritation, and flushing fluid through the catheter. Patient comfort is improved by decreasing pressure on the trigone, and as well the ability of the material to adapt to the curvature of the urethra. Blockage is reduced as a result of the plurality of drainage channel/chambers created by the bundle of fiber arrangement.

It will be appreciated that many of the features described above are novel in their own right, at least in the field of catheter devices. These, other features and advantages will be more apparent with description of embodiments with reference to drawings and figures.

The invention will now be described in more detail with objectives and advantages while viewed with the following drawings:

### BRIEF DESCRIPTION OF THE DRAWINGS

FIG. 1A Fibers/filaments formation of channel.

FIG. 1B Fiber arrangement in bundles.

FIG. 2 Side cross-sectional view of the present invention showing connectors, ports and collection attachment.

FIG. 3A Side cross-sectional view of outer sleeve connector.

FIG. 3B Axial cross-sectional view of outer inner sleeve connector.

FIG. 3C Side cross-sectional view of the activation of the outer sleeve connector released from the inner sleeve.

FIG. 4A Longitudinal cross-sectional view of the resistant anchoring system of the fibers/filament material in the inner sleeve.

FIG. 4B Side cross-sectional view of the activation of the inner sleeve.

FIG. 5A Side cross-sectional view of shield secured by attachment clip.

FIG. 5B Side cross-sectional view of clamp device securing mechanism.

FIG. 5C Side cross-sectional view of hub and groove securing mechanism.

FIG. 5D Side view of attachment clip.

FIG. 6A Side view of external shield.

FIG. 6B Top view of external shield.

FIG. 6C Side view of internal shield.

FIG. 6D Top view of internal shield.

FIG. 7 Side cross-sectional view showing the removal of the outer sleeve out of the urethra and top of the inner sleeve.

FIG. 8 Enlarged side cross-sectional view of completely inserted catheter showing the fibers/filaments material weaved or wrapped around the inflated balloon inside the urethra and bladder in an upright patient.

FIG. 9 Catheter for irrigation.

FIG. 10 Catheter for chest tube applications, or wound drainage.

The figures are purely schematic and are not drawn to scale. In cases of clarity the dimensions may be exaggerated to a greater or lesser extent.

### DETAILED DESCRIPTION OF INVENTION OF THE PREFERRED EMBODIMENT

The terms proximal and distal refer to the position of the patient while the catheter is in use. Proximal refers to positions near the patient while distal refers to positions away from the patient.

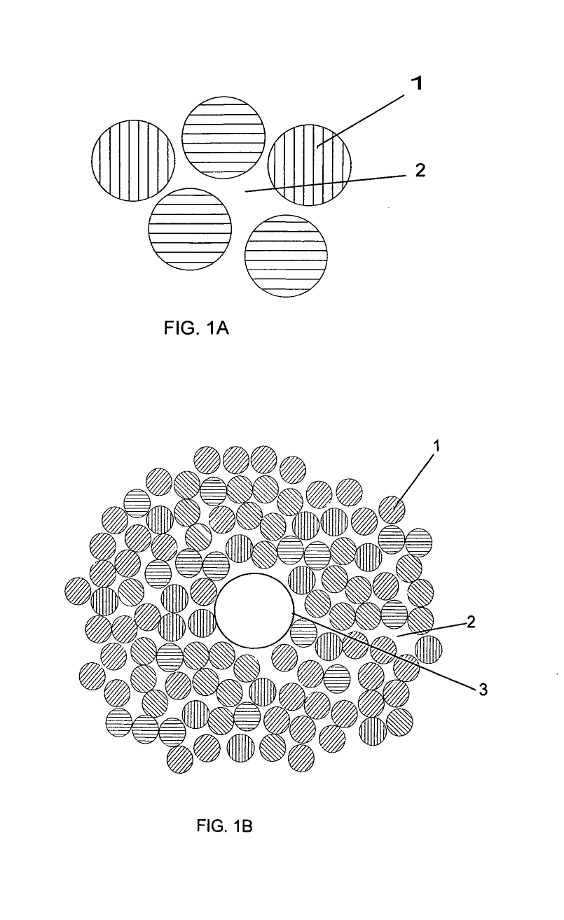

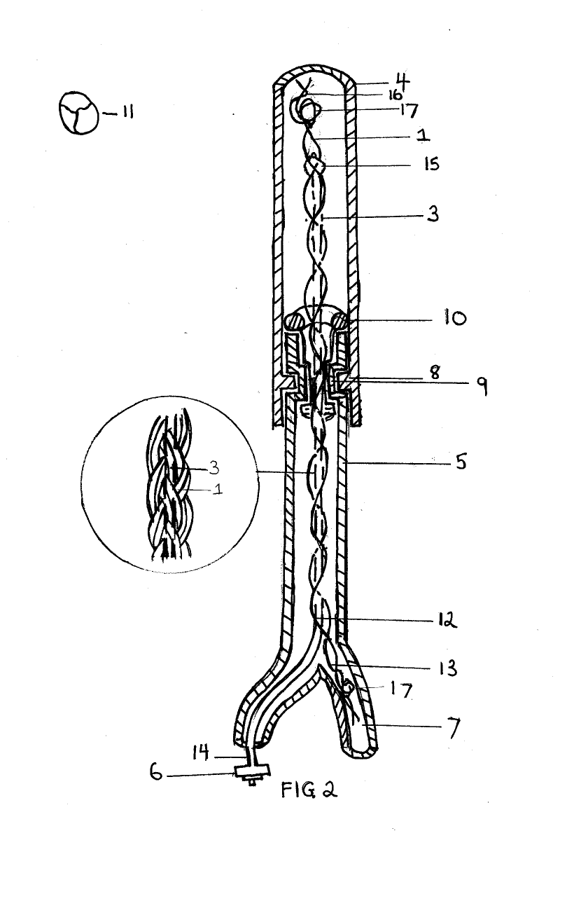

As illustrated in FIG. 1A, an arrangement of fibers 1 creating channels 2 for movement of fluid. FIG. 1B shows how the arrangement of fibers braided together around a very fine tube of the inflation device 3. The inflation device may be of conventional construction and braided around with flexible fibers. The structure of fibers can vary in size and orientation so that fluid flow occurs. The essential element of the catheter is the flexible fibers formation of a plurality of channels to provide fluid movement.

The parts are manufactured from a biocompatible material to avoid excessive irritation to the body. The tube, flexible fibers, and inflation device can be constructed of natural rubber, latex, polyvinyl chloride, polyurethane, polytetrafluorethylene, silicone rubbers, nylon or other similar material.

In FIG. 2 the catheter is comprised of an elongated tube with an outer 4 and inner sleeve 5 which encloses the fibers 1 that are braided, wrapped or weaved around a central inflation device 3 with connectors for inflation 6, and drainage/irrigation 7. The fibers that are braided around the central inflation device extends through the interior of the catheter. The outer 4 and inner 5 sleeves form the exterior of the flexible tubular catheter that enclose the inner length of the flexible fibers 1. The outer sleeve 4 partially encloses the flexible fibers 1, and interlocks with the inner sleeve 5 via the projections 8. The outer 4 and inner 5 sleeve are preferably arranged with a significant degree of separation between the tubular walls, making it possible for movement of the outer sleeve 4 over top the inner sleeve to expose the flexible fibers 1 and inflation device 3. The flexible fibers 1 which are braided around the inflation device 3 is contained within the two sleeves 4,5 and thereby remains free from contamination when handled for insertion. At the end of the outer sleeve 4 it is optional to have a breakable seal 11 to further enhance the sterility of the device. Fluid entering the catheter at the proximal end of the fibers 1 will exit at the distal end in a collection reservoir. The inflation device 3 and fibers 1 separate near the distal end with the fibers ending a distance from the collection port. Fluid movement continues along the fibers channels 2 as a result of the surface tension of the fluid. At the distal end of the inner sleeve 5 a length of the inflation device 3 is left outside to accommodate for adjustment of the catheter. As with the inflation device, a length of the fibers 1 is used to accommodate for adjustment, however, the fibers are still contained within the inner sleeve 5. The fibers are prevented from fraying by heat sealing the ends, clamping ends, using a knot 16, or other means to stabilize fraying near the end of the fibers distal and proximal ends. The inner sleeve 5 may contain an attachment point for a collection bottle/irrigation port 7, and an inflation port 6 for injecting saline, water or air which activates the retention balloon 15. The inner sleeve 5 is hollow along its full length in order to provide space for the fibers 1 and inflation device 3 thus providing a drainage guide along which fluid can move. At the distal end, the inner sleeve bifurcates 12 providing an attachment point for a syringe 6 used to inflate the balloon, and a connector for a fluid storage reservoir/irrigation port 7. At the distal end, a drainage tube is provided with fittings enabling the catheter to be connected to an appropriate drainage conduit, reservoir, or the like. In the present invention the drainage conduit may also be used for irrigation.

An advantage of the current invention is the elimination of residual urine. A length of fibers 16 extends past the retention balloon 15 and may lay on the floor of the bladder where fluid can drain by capillary action. Drainage of fluid with this catheter is not dependent on the positional state of a patient, since the capillary movement of fluid can work against gravity to drain residual urine from a patient in any position. In other catheters optimal drainage occurs with the patient is only in the upright position.

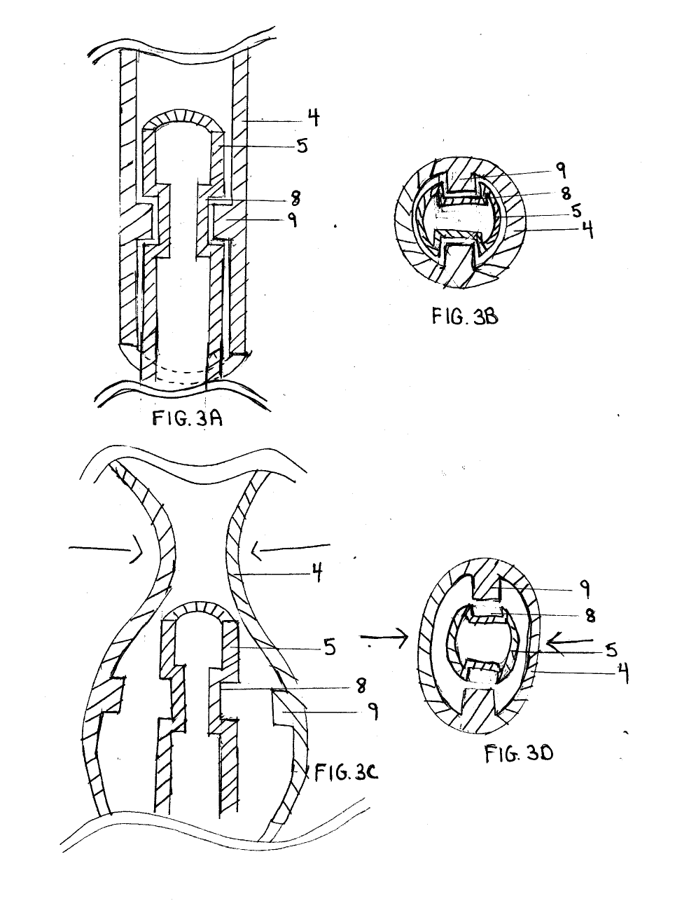

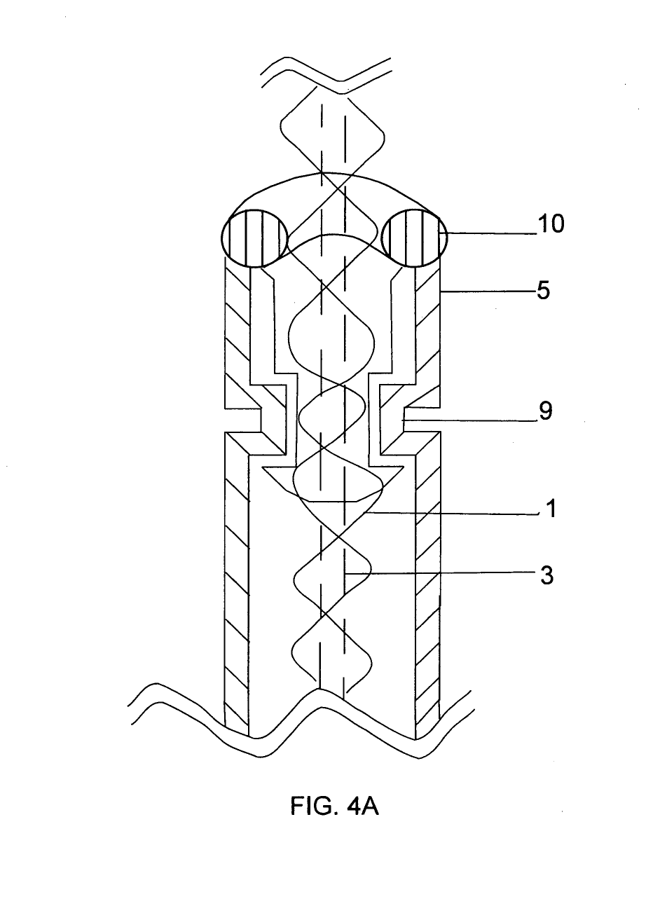

The proximal portion of the inner sleeve 5 is inserted into the distal portion of the outer sleeve 4, as shown in FIG. 3A,B. The connection between the inner 5 and outer sleeve 4 is composed of two grooves 9 and projections 8 locate adjacent to one another. The sleeves are made of a flexible resilient or elastic structure allowing for distortion FIG. 3C,D that creates an lock and release system between inner 5 and outer 4 sleeves. This connection is composed of interlocking projections 8 and grooves 9 partially encompassing the diameter of the tube. In FIG. 3C, D projections 8 and grooves 9 are constructed along a portion of the circumference of the tubular sleeves 4,5. This creates a secure fit but can be easily opened when pressure is applied to the sides adjacent the connection or interlock FIG. 3C, D. Pressure causes the flexible tube to change conformation and open the connection, allowing the outer 4 and inner 5 sleeves to move. Removal of the outer sleeves 4 exposes the fibers 1 and inflation device 3 for use. There is no need to handle the fibers 1 and inflation device 3 of the catheter during insertion into the body cavity. This is a favorable advantage of the present catheter as cleanliness and sterility is maintained.

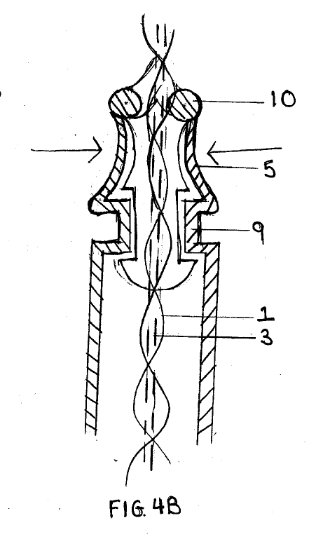

In FIG. 4A the inner sleeve 5 of the catheter anchors or fixes the fibers 1 and inflation device 3 to the inner sleeve 5. The inner sleeve 5 is composed of two projections 9 along with the shield 10 which is used to secure the fibers 1 and inflation device 3 thereby limiting movement of the fibers 1 within the inner sleeve 5. Relief of the fibers 1 and inflation device 3 FIG. 4B is accomplished by pressing its sides causing a change in conformation of the flexible tube which releases the interlock of projections 9 enabling movement.

The connection between the inner 5 and outer 4 sleeves has three uses. First, when the inner 5 and outer 4 sleeves are interlocked it provides a rigid tube for insertion into a body cavity. Second, the inner sleeve grooves/projections 9 are designed to stabilize or secure the fibers 1 within the inner sleeve 5 and allow for positioning. Lastly, the inner sleeve grooves/projections 9 provide an attachment point for a silicone cover that prevents irritation between the outside of the inner sleeve 5 and the patient. The inner sleeve grooves/projections 8, functions as grooves for connection with the outer sleeve projections, and act as projections for securing the fibers 1 and inflation device 3.

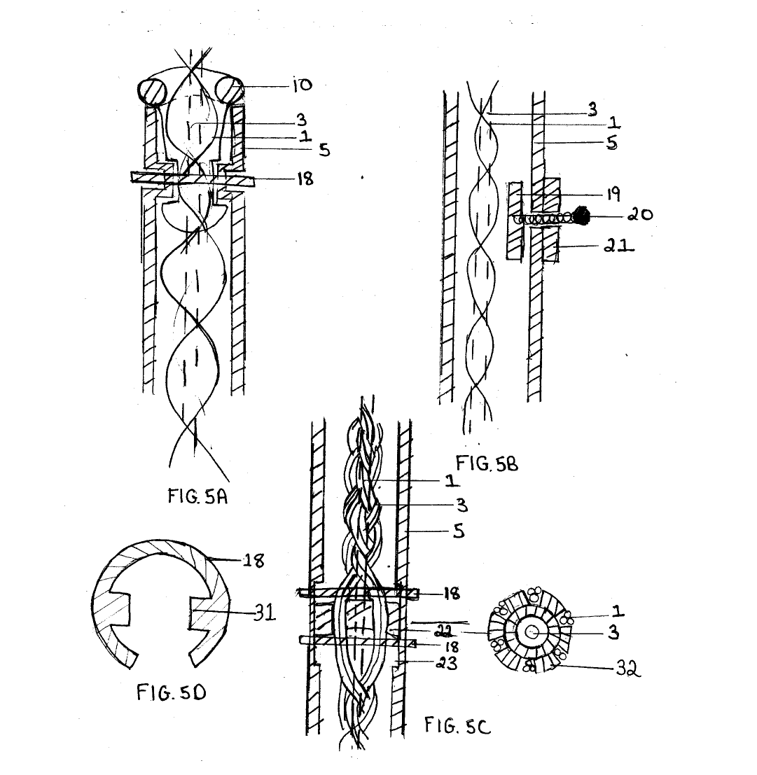

Other arrangements of securing the braided fibers 1 and inflation device 3 to the inner sleeve 5 are illustrated in FIG. 5A, B, C, D. The shield device arrangement may be further secured with the use of a clip 18 which fits over the outside of the inner sleeve 5 and increases the constrictive pressure on the fibers 1 and inflation device 3 FIG. 5A. This arrangement will help prevent accidental activation and release of the inner sleeve 5 over the fibers 1 and inflation device 3. In FIG. 5B fibers 1 and inflation device 3 are secured to the inner sleeve 5 with a clamp 19 that applies pressure through activation of turns by a threaded screw 20 and reinforcing bar 21 located on the inner sleeve. This device is located further down the inner sleeve 5 to avoid conflict with the outer sleeve 4 when it is pulled out of the body cavity. In FIG. 5C a secure fix of the fibers 1 and inner sleeve 5 is accomplished by the use of a hub 22 containing projections that are interlaced with the fibers 1 while the inflation device 3 passes through the center of the hub 22. Grooves 23 are contained around the diameter of the inner sleeve 5 wall in which the hub 22 may be located and move to accommodate adjustments. Once the desired position is achieved two clips 18 are place on either side of the hub 22 to secure it from movement. FIG. 5D is a schematic representation of the clip 18 with projections for securing the fibers 1 and inflation device 3.

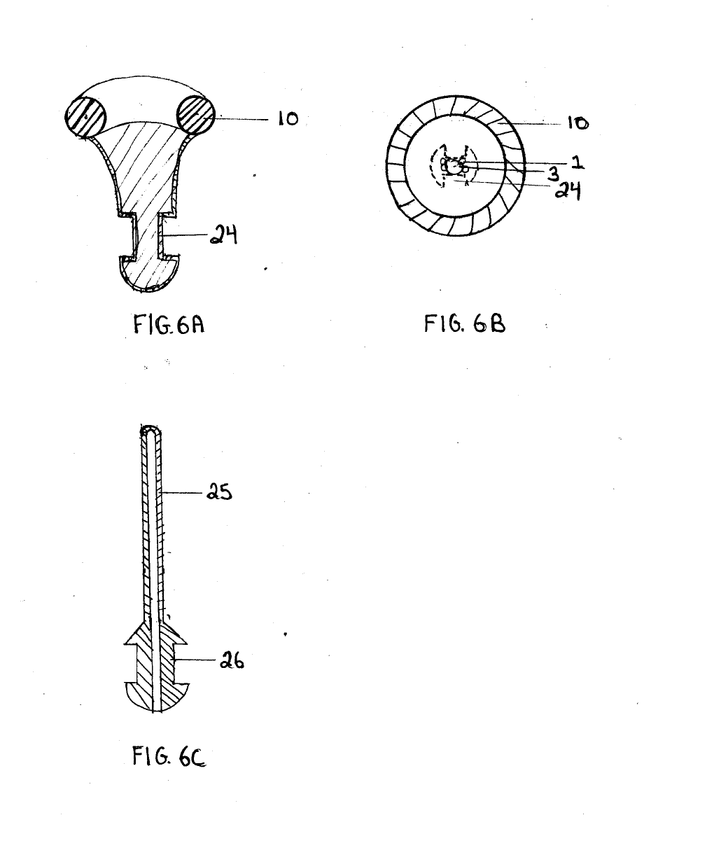

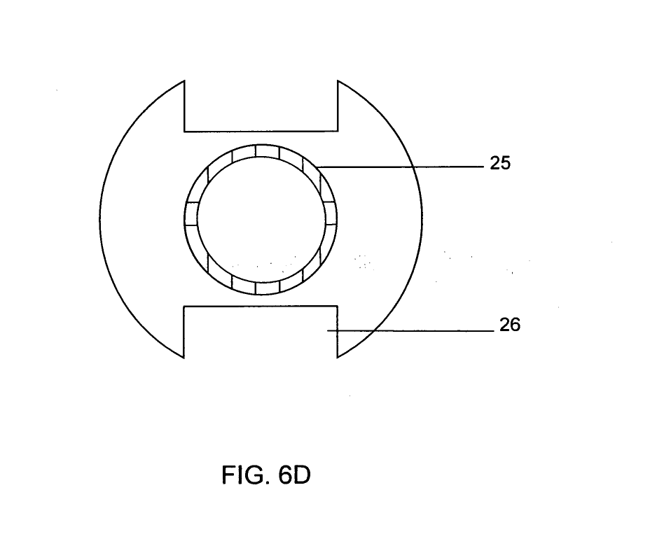

The shield shown in FIG. 6A,B,C,D may be configured in two different arrangements to prevent body structure irritation with the top of the inner sleeve 5, and the seepage of fluid by outside contact of the fibers 1. Both are secured in placed by grooves 24,26 which interlock with grooves/projection 9 on the inner sleeve 5. One shield arrangement is positioned outside the body while the other shield arrangement is designed to be inserted into a portion of the urethra. In the former arrangement the shield may also function to prevent leakage of fluid between the inner 5 and outer 4 sleeve during irrigation of the bladder, and to funnel large masses of fluid and debris through the irrigation port. In the later arrangement a portion of the tube 25 is located within the urethra to prevent irritation and seepage of fluid outside the catheter. In this arrangement the fibers 1 and inflation device 3 are contained within the tube structure. This shield configuration may hinder the flow of irrigation solution because of the narrow opening.

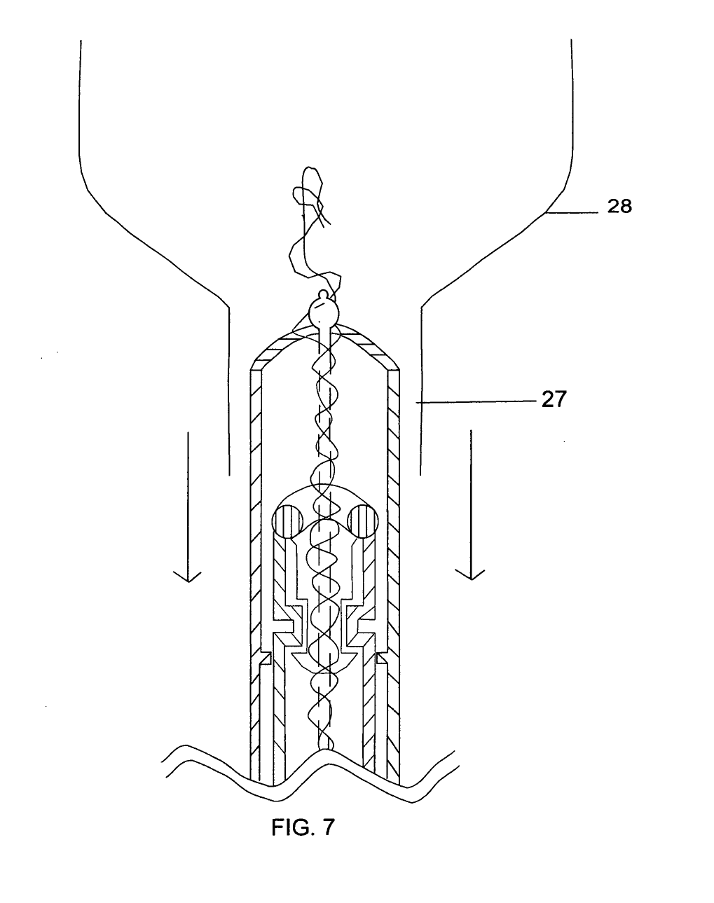

FIG. 7 illustrates removal of the outer sleeve 4 after the insertion of the catheter in to the urethra 27 and bladder 28. The outer sleeve 4 has rounded edges which help in the insertion of the catheter into the urethra 27. Once fluid is detected entering the catheter the catheter is further inserted an appropriate distance to ensure the retention balloon resides inside the bladder 28. The balloon is then inflated and the remaining portion of the outer sleeve is removed over top of the inner sleeve. When the outer sleeve 4 has been inserted fully into the urethra 27, pressure on the outer sleeve connector releases the interlock connection and allows the outer sleeve 4 to move over top of the inner sleeve 5 and out of the urethra 27. This is accomplished while the inner sleeve 5 is held in place by hand.

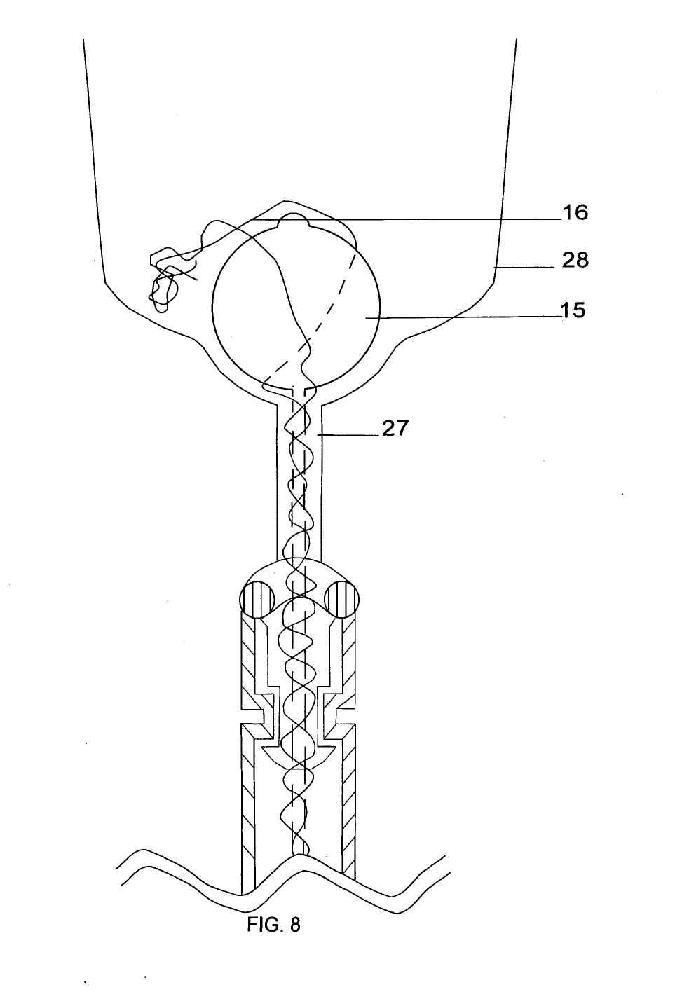

As illustrated in FIG. 4A, B once the interlock connection is released the inner sleeve 5 is movable over the fibers 1. This is accomplished because the inner components of the catheter, braided around an inflation device, are not fixed to the inner sleeve 5. The fibers separate from the inflation device in a Y like pattern at the distal end of the catheter as shown in FIG. 2. The inflation device 3 exits the fibers through part of the braid. There is excess inflation device 3 tubing at the distal outside end of the catheter's inner sleeve 5 to allow for its positioning movement. The outer sleeve 4 may be removed over the entirety of the inner sleeve 5 or may remain stored on the inner sleeve 4, for later use in irrigation. Once the catheter is inserted into the bladder 28, fluid enters the collection port 7 and drains from the catheter, indicating to the user that the retention device 15 is ready to be deployed. The outer sleeve is then withdrawn to reveal the sterilized, flexible fibers/filaments material containing the retention balloon in the bladder. With one hand firmly placed on the inner sleeve 5, the outer sleeve 4 is pulled back to an appropriate distance on the outside of the inner sleeve 5. Here, the retention balloon 15 is inflated, leaving a length of flexible braided fibers 16 in the bladder as revealed in FIG. 8. The outer sleeve 4 could be removed entirely from the catheter assemble, or the outer sleeve 4 could be pulled back over top of the inner sleeve 5 to a point where it reveals the proximal end of inner sleeve 5. At this point the outer sleeve 4 may be connected to the top of the inner sleeve 5. The invention creates a method of reducing infection by inserting sterilized nylon fibers 1 into the bladder 28. As opposed to thick walled catheters, this catheter with it arrangement of flexible fibers 1 can be easily moved and bent within the urethra 27, during insertion and normal patient activities. The flexible fibers 1 such as nylon would provide for increase comfort compared to known catheters which consist of more rigid tubular material. Contact with the trigone area of the bladder is decreased by the presence of the braided fibers 1 encompassing the retention balloon 15, this structure mimics the wing like structured used by other catheters to reduced the pressure of a balloon on the trigone region of the bladder. The constriction caused at the neck of the bladder as a result of balloon inflation and the urethra sphincter will have minimal impact on fluid drainage, since there is a plurality of channels by which fluid can pass. As disclosed the retention balloon 15 can facilitate anchorage, it is also apparent that anchoring can also be achieved with other structures. Because of the flexibility and simplicity in use and insertion, this catheter would help reduce infection, prevent catheter blockage, more completely eliminate urine, and improve comfort for the patient.

Catheters are normally pre-packaged in a receptacle for sterilization and protection of the device. Urinary catheters generally need to have lubricant applied to the outer surface to facilitate insertion into the urethra. Any water-based lubricant usually works. In other embodiments, the urinary catheter fibers are designed to be pre-lubricated. Pre-lubrication can be accomplished by injecting the lubricant into the outer sleeve and coating the braided fibers.

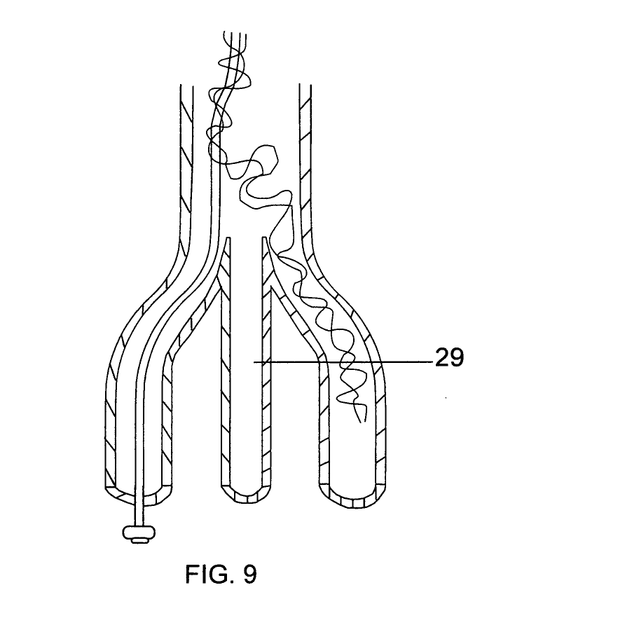

In one embodiment FIG. 9 the insertion of the current catheter into the anatomical region introduces a fluid under pressure into the proximal end of the lumen of the irrigation port 29 for enhanced fluid irrigation of the bladder after prostate or bladder surgery. By using the entire catheter assembly, with movement of the outer sleeve 4 to a position that allows for the inflation of the retention balloon 15, the catheter can be used for irrigation. The shield 10 in the connection joint will prevent any leakage of fluid and funnel the fluid down the inner sleeve 5. Since the area of projections 9 and shield grooves 24 are not continuous around the sleeves, this will allow for passage of larger material.

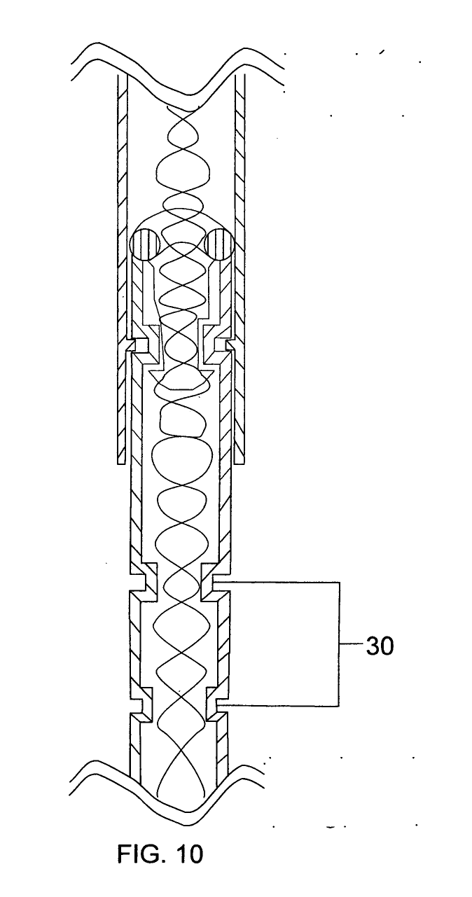

In one embodiment of the invention, the catheter of the invention can be fabricated without the central inflation device 3 containing the retention balloon 15. This catheter illustrated in FIG. 10 can be used to drain pleural space or an abscess or remove fluid from other body cavities. When modified, the current invention can be used as a chest tube drainage catheter. In this configuration, air can be expelled from the chest cavity while fluid continues to drain. Once inserted the outer sleeve 4 can be adjusted with varies grooves 30 located on the inner sleeve 5 for positional adjustment of the catheter. The current invention is an advance over other chest tube catheters as it reduces the chance of blockage. When blocked, conventional catheters require the use of TPA (alteplase) to break down the clot. The current invention decreases the chance that TPA may be need, and can be a significant financial savings for health care providers.

The present disclosure is not limited to a urinary catheter, but includes other types of drainage catheters for chest drainage or wound drainage as shown in FIG. 10. In FIG. 10 the device being modified to support a chest tube and wound drainage applications without the retention device and a threaded end that attaches to a collection vessel.

The present invention is concerned with modification to the lumen of the catheter so as to provide a prolonged period over which the catheter can be left in position in the body before onset of problems mentioned above.

The embodiments of the invention in which an exclusive property or privilege is claimed are defined as follows:

1. A catheter for drainage of a body cavity, comprised of elongated tubular sleeves, an outer and inner sleeve, sized for insertion into a body cavity; having a proximal outer sleeve end for insertion in said body cavity and a distal inner sleeve end adapted to be connected to a drainage conduit, reservoir or the like; having flexible fibers/filaments encompassing an inflation retention member contained within the elongated tubular sleeves.

2. The catheter recited in claim 1 consisting of flexible fibers/filaments forming a plurality of drainage channels for movement of fluid.

3. The catheter recited in claim 1 wherein said channels are formed by fibers/filaments having varying range in diameter or length.

4. The catheter recited in claim 1 wherein length of flexible fibers/filaments extends past the retention balloon through which fluid can pass from the body cavity into a reservoir.

5. The catheter recited in claim 1 containing flexible fibers/filaments material for insertion into a canal or canal-like organ to facilitate drainage of fluid.

6. The catheter recited in claim 1 wherein the spacing of the inner sleeve from the patient tissue is such as to prevent rubbing or irritation by a funnel-like shield.

7. The catheter recited in claim 1 wherein proximal to the inner sleeve is material for protection of irritation. Encompassing an elongated arrangement to prevent outside contamination, irritation and seepage of fluid outside the catheter.

8. The catheter recited in claim 1 wherein the flexible fibers/filaments can be anchored in the bladder by different mechanisms.

9. The catheter recited in claim 1 wherein the flexible fibers/filaments may be impregnated or coated.

10. The catheter recited in claim 1 wherein the flexible fibers/filaments may be impregnated with material of antimicrobial action.

11. The catheter recited in claim 1 wherein the flexible fibers/filaments may be coated with Teflon.

12. The catheter recited in claim 1 wherein the catheter is made of biocompatible material.

13. The catheter recited in claim 1, wherein a closed package accommodates said catheter before being opened for the purpose of sterilization.

14. The catheter recited in claim 1 wherein the flexible fibers/filaments and inflation device are secured by the inner sleeve projections, and or clip, or with a clamp-like device.

15. The catheter recited in claim 1 wherein the flexible fibers/filaments and inflation device are connected or positioned by use of projections and grooves.

16. The catheter recited in claim 1 for insertion into the urethra to facilitate the drainage of urine from the bladder, comprising a flexible tubular body defining an outer sleeve and inner sleeve mateable by a connection for fixed positioning the catheter.

17. The catheter recited in claim 1 wherein the separation of outer sleeve from the inner sleeve wherein said apparatus is configured such that the proximal end of said catheter moves toward said distal end portion, thereby allowing insertion of non-contaminated flexible fibers/filaments material. Once inserted the distal end of the catheter may move when pinch type connector is activated.

18. A catheter comprising an outer sleeve connected to an inner sleeve while anchored within are fibers/filaments to direct fluid from the body to a collection vessel.

19. A catheter for drainage of a body cavity, said catheter comprising elongated tubular sleeves being sized for insertion into a body cavity; having a proximal outer sleeve end for insertion in said body cavity and a distal end adapted to be connected to a drainage conduit, irrigation port, reservoir or like. Contained within flexible fibers/filaments forming a plurality of channels for fluid drainage. Wherein the catheter's proximal outer sleeve end allows for non-contaminated insertion of flexible fibers/filaments.

20. The catheter recited in claim 19 for adaptation as urinary catheter, chest tube or wound drainage.
